(19) United States
(12) Patent Application Publication
(10) Pub. No.: US 2008/0297169 A1
(43) Pub. Date: Dec. 4, 2008

(54) Particle Fraction Determination of A Sample

(76) Inventors: Alfred C. Greenquist, San Jose, CA (US); Margaret McInerny, Monkstown (IE)

Correspondence Address: Inverness Medical Innovations / WSGR;Wilson Sonsini Goodrich & Rosati, P.C., 650 Page Mill Road, Palo Alto, CA, 94304, US

(21) Appl. No.: 11/756,582

(22) Filed: May 31, 2007

(51) Int. Cl. G01N 33/86; B01J 19/00; G01N 27/06; G01N 27/07

(52) U.S. Cl. 324/600; 422/68.1; 422/73; 422/82.01; 436/150; 436/69

(57) ABSTRACT

The present invention provides a device, test cards, methods and kits which are useful for determining the particle fraction and rate of viscosity of a fluid sample, the presence of an analyte in a fluid sample, or the aggregation of particles in a fluid sample to detect an analyte or as an immunologic assay.

### BACKGROUND OF THE INVENTION

Field of the Invention: This invention relates to analytic systems, devices and methods for determining the condition of a fluid sample; more particularly to measuring the fraction of a suspension that constitutes the solid, or particulate, portion of the total volume element. More specifically, this invention relates to systems using a disposable test card configured to provide sensors adapted for use in conjunction with a detection device for the measurement of impedance of a fluid sample that contains analytes and/or particles, such as blood or blood components or microbes. Such devices can be used, e.g., for the accurate determination of analytes, change in viscosity, particle fractions, and aggregated particles for analytical, hematologic, immunologic, and microbiologic assays.

Background: Testing of body fluids is useful for medical diagnosis of disease or pathologic states, or to monitor the effect of therapeutics and treatments in diverse applications. Therapeutic intervention with anticoagulants and lysis agents including antithrombins and antiplatelet agents are often used to manage patients with thrombotic disease. As a component of the monitoring of such patients, various hemostasis tests have been devised, including modifications of the prothrombin time test. Similarly, patients with anemias or bleeding disorders are often monitored by tests for the amount or function of various blood cells. Also, the immune status of a patient may be monitored by the use of assays designed to detect specific analytes, immunoglobulins, cytokines, cell receptors, foreign particles such as microbes, and other blood or body fluid factors.

Testing can be performed on a variety of body fluids, but is most routinely performed on whole blood, plasma, or serum, urine and cerebrospinal fluid. Whole blood contains a particle phase that has cells in varying quantities, including erythrocytes, white blood cells, and platelets. Blood also has a liquid phase, i.e., plasma, which contains proteins that, amongst other properties, serves to coagulate blood to control bleeding, transport nutrients, hormones and minerals, and contains immunoglobulins and other factors to mediate an immune response.

Various methods and devices have been employed for assaying fluids that contain particles, including devices that perform determinations by optical, mechanical, and impedance detection means. The hematocrit assay determines the particle fraction of erythrocytes of whole blood and can serve as an estimate of hemoglobin and amount of erythrocytes in the circulating blood. The coagulation rate measures the ability of blood proteins to clot whole blood. The aggregation of platelets measures the functional ability of these blood cells to initiate a clot. Modernly, analyte and immunologic assays have been devised that utilize the aggregation of cells or artificial particles that are complexed with chemical or immunologic reagents to determine the desired immunologic analyte or parameter in a body fluid.

In disease, pathologic, or therapeutic conditions where assays must be repeated at frequent intervals, the ability to perform multiple assays with small amounts of blood is preferred. Additionally, assay devices that are portable and use disposable sample cards permit ease of use by the patient or at point of care in a doctor's office, and increase safety.

Accordingly, there remains a need for a test system and methods in which multiple assays can be reliably and quickly performed using single use, disposable test cards that require small amounts of blood and can be used in a self-contained portable device to provide accurate assay results.

### SUMMARY OF THE INVENTION

The present invention generally relates to sample analyzer systems, devices and methods for determining the condition of a fluid sample. More particularly, the sample analyzer system of the present invention provides a device and methods for performing multiple assays on fluid samples, including blood, plasma, serum, urine, cerebrospinal or other body fluids, as well as non-bodily fluids.

The analyzer system of the present invention provides methods, test cards and a device for performing particle fraction, changes in viscosity, cell aggregation, analyte and immunologic assays on a fluid sample based on the electrical impedance of the sample. More particularly, the analyzer system of the present invention applies an AC or DC electric potential to the fluid sample and detects impedance, resistance, change of impedance, change of resistance, rate of change of impedance or rate of change of resistance of the fluid sample as a component of the assay methods.

The present invention further provides kits containing the device, test cards, and instructions for using the assay methods of the present invention.

The present invention provides a test card adapted for use by the device for determining the particle fraction, change in viscosity, presence or amount of an analyte, rate of coagulation or lysis rate, or the particle aggregation of a fluid sample. The test card includes a laminate substrate defining a surface for receiving the sample and micro-fluidic channels for delivering a defined volume of fluid by capillary action to two or more assay chambers, each containing two electrodes positioned on the substrate for contacting the sample. The electrodes are adapted to receive and pass a predetermined electric potential into the sample that is detected by the device of the invention. The electrodes generate and detect an electrical signal corresponding to the impedance or resistance of the sample, which is processed by the device of the invention into assay results.

The present invention also provides methods of determining a particle fraction of a fluid sample by applying a potential to the sample, which is used to determine the hematocrit or the aggregation of blood, microbial or synthetic particles for hematological, immunological, or analyte assays by measuring the impedance, resistance, net change in impedance or net change in resistance in comparison to stored assay calibration information. The invention further provides methods of determining changes in the viscosity of the fluid sample, such as the rate of coagulation or lysis of a fluid sample. For example, a rate of coagulation assay of the invention, which includes the steps of: accelerating coagulation of the sample by chemically reacting the sample with at least one reagent to produce a detectable change in the impedance or resistance of the sample which correlates with a state of coagulation or lysis of the sample; measuring the rate of change in impedance or resistance of the sample and generating a signal which correlates to a curve of the coagulation or lysis; and processing the signal into an output corresponding to the coagulation or lysis assay using assay reference calibration information.

In one embodiment, a method for determining a particle fraction of a fluid sample is provided. The method includes the step of: measuring impedance or resistance of said fluid sample, wherein said fluid sample volume is up to 20 μL. Impedance or resistance can be measured, for example, by applying an AC or DC electric potential to the fluid sample. Additionally, impedance or resistance can be correlated to a calibration standard curve to produce an impedance or resistance correlation, which can then be further processed into an output result. Sample fluids can include body fluids, such as blood, non-bodily fluids, and water.

In another embodiment, the invention is directed to a method for determining particle fraction of a fluid sample comprising the step of: applying said fluid sample to a chamber having volume up to 2 μL; and measuring an impedance or resistance of said fluid sample in said chamber. Impedance can be obtained by, for example, applying an AC electric potential to the fluid sample. Similarly, resistance can be obtained by, for example, applying a DC electric potential to said sample. Additionally, impedance or resistance can be correlated to a calibration standard curve to produce an impedance or resistance correlation, which can then be further processed into an output result. Sample fluids can include body fluids, such as blood, non-bodily fluids, and water.

In yet another embodiment, the invention encompasses a method for determining particle fraction and viscosity of a fluid sample comprising: applying said fluid sample to a chamber having volume up to 2 μL; and measuring impedance or resistance of said fluid sample in said chamber. Impedance can be obtained by, for example, applying an AC electric potential to the fluid sample. Similarly, resistance can be obtained by, for example, applying a DC electric potential to said sample. Additionally, impedance or resistance can be correlated to a calibration standard curve to produce an impedance or resistance correlation, which can then be further processed into an output result. Sample fluids can include body fluids, such as blood, non-bodily fluids, and water. In some embodiments, the measuring step of the method can further comprise: measuring impedance or resistance at one or more time points of said fluid sample and correlating said impedance or resistance to a particle fraction calibration standard curve to produce a first impedance or resistance correlation; measuring rate of change of impedance or resistance over time of said fluid sample and correlating said impedance or resistance to a coagulation calibration standard curve to produce a second impedance or resistance correlation; and processing said first and second impedance or resistance correlations into output results corresponding to the particle fraction and viscosity of said fluid sample. Suitable reagents include reagents for prothrombin time, activated clotting time, activated partial prothrombin time, or thrombin clotting time.

In still another embodiment, the invention is directed to a method of determining particle fraction and viscosity of a fluid sample comprising the steps of: applying said fluid sample to a first chamber having volume up to 2 μL and a second chamber having volume up to 2 μL wherein the first chamber further comprises one or more reagents for accelerating a coagulation of a fluid sample upon contact with said fluid sample; measuring impedance or resistance of said fluid sample at one or more time points in the second chamber and correlating the impedance or resistance to a particle fraction calibration standard curve to produce a first impedance or resistance correlation; measuring a rate of change of measured impedance or resistance over time of said fluid sample in said first chamber and correlating said impedance or resistance to a rate of coagulation calibration standard curve to produce a second impedance or resistance correlation; and processing said first and second impedance or resistance correlations into output results corresponding to a particle fraction and viscosity of said fluid sample. Impedance can be obtained by, for example, applying an AC electric potential to the fluid sample. Similarly, resistance can be obtained by, for example, applying a DC electric potential to said sample. Additionally, impedance or resistance can be correlated to a calibration standard curve to produce an impedance or resistance correlation, which can then be further processed into an output result. Sample fluids can include body fluids, such as blood, non-bodily fluids, and water.

In yet another embodiment of the invention, a method for determining the amount or presence of analyte in a fluid sample is provided. The method comprises the steps of measuring change of impedance or resistance of said fluid sample in a chamber wherein chamber comprises a binding moiety that selectively binds to said analyte and wherein volume of said chamber is up to 2 μL. In some embodiments, the determining step can further comprise: applying AC or DC electric potential to said chamber; measuring impedance or resistance at two or more intervals; correlating said change of impedance or resistance to an analyte calibration standard curve; and processing said impedance or resistance correlations into output results corresponding to the amount or presence of said analyte of said fluid sample. Analytes can be, for example, mammalian cells, microbial cells, drugs, chemical compounds, hormones, proteins, and immunoglobulins, to name a few. Additionally, the binding moiety can be immunoglobulins, monoclonal antibodies, avidin, compromised avidin, streptavidin, lectins, protein A, haptens, biotin, iminobiotin, or sugars. As will be appreciated, in some embodiments, the binding moiety can be coupled to a substrate, such as latex beads, whole blood cells, erythrocytes, white blood cells, platelets, colloidal gold particles, magnetic particles, quantum dots, bacteria, viral particles, or liposomes.

In another embodiment, a method for determining the amount or presence of analyte in a fluid sample is provided, wherein said determining step comprises: applying said fluid sample to two chambers, said chambers having volume up to 2 μL, wherein a first chamber comprises a binding moiety for agglutinating an analyte in a fluid sample upon contact with said fluid sample and a second chamber does not comprise said binding moiety; applying AC or DC electric potential to said first and second chambers; measuring the difference in impedance or resistance of said fluid sample in said first and second chambers at one or more intervals; correlating said difference in impedance or resistance to an analyte calibration standard curve to produce an impedance or resistance correlation; and processing said impedance or resistance correlation into output results corresponding to the amount or presence of said analyte of said fluid sample. Analytes can be, for example, mammalian cells, microbial cells, drugs, chemical compounds, hormones, proteins, and immunoglobulins, to name a few. Additionally, the binding moiety can be immunoglobulins, monoclonal antibodies, avidin, compromised avidin, streptavidin, lectins, protein A, haptens, biotin, iminobiotin, or sugars. As will be appreciated, in some embodiments, the binding moiety can be coupled to a substrate, such as latex beads, whole blood cells, erythrocytes, white blood cells, platelets, colloidal gold particles, magnetic particles, quantum dots, bacteria, viral particles, or liposomes.

The invention is also directed to devices for determining particle fraction, amount or presence of an analyte, and viscosity of a fluid sample. The devices comprise a detection unit adapted and configured to apply AC or DC electric potential to said fluid sample and measure one or more impedance or resistance signals of said fluid sample; and a processor unit electrically connected to said detection unit adapted and configured to receive and convert said impedance or resistance signals into output results corresponding to said particle fraction, amount or presence of said analyte, and viscosity of said fluid sample. The processor unit can be adapted and configured to separately determine said particle fraction, amount or presence of said analyte, or viscosity of said fluid sample from said impedance signals using assay calibration standard curve information stored in the processor. Particle fractions can be, for example, hematocrit, or aggregated particles. Analytes can be, for example, mammalian cells, microbial cells, drugs, chemical compounds, hormones, proteins, and immunoglobulins.

Another invention is directed to a test card for determining a condition of a fluid sample comprising: an inlet port for receiving a fluid sample of less than 20 μL fluidly connected to two or more capillary channels wherein each capillary channel terminates in a fixed volume chamber having a volume up to 2 μL. In some embodiments, each of said chambers can be further adapted to comprise two electrodes for measurement of impedance or resistance in said fluid sample. Additionally, one or more said chambers can further comprise one or more reagents for accelerating the coagulation of said fluid sample upon contact with said fluid sample and/or one or more binding moieties that selectively binds to an analyte. Analytes can be, for example, mammalian cells, microbial cells, drugs, chemical compounds, hormones, proteins, and immunoglobulins, to name a few. Additionally, the binding moiety can be immunoglobulins, monoclonal antibodies, avidin, compromised avidin, streptavidin, lectins, protein A, haptens, biotin, iminobiotin, or sugars. As will be appreciated, in some embodiments, the binding moiety can be coupled to a substrate, such as latex beads, whole blood cells, erythrocytes, white blood cells, platelets, colloidal gold particles, magnetic particles, quantum dots, bacteria, viral particles, or liposomes.

The advantages, embodiments, variations and the like will be apparent to those skilled in the art from the present specification taken with the accompanying drawings and appended claims.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a test card in combination with a measuring device.

FIGS. 2A-C illustrate a process for fabricating a test card.

FIGS. 3A-B illustrate a top view of two configurations of a test card.

FIG. 4 illustrates a flowchart for exemplary methods of the assay system.

FIG. 5 illustrates a kit of the present invention.

FIG. 6 illustrates the plot of impedance vs. hematocrit, derived from results of Example 1.

FIG. 7 illustrates the plot of log impedance vs. hematocrit, derived from results of Example 1.

FIG. 8 illustrates the plots of impedance vs. hematocrit in samples obtained from a Coumadin-treated patient and an untreated subject, derived from results of Example 1.

FIG. 9 illustrates the comparison of plots of log impedance vs. hematocrit from samples measured at three different time intervals in the same chamber, derived from results of Example 1.

FIG. 10 illustrates plots of impedance vs. hematocrit from samples tested in chambers without coagulation reagents from results of Example 1.

FIG. 11 illustrates the comparison of plots of log impedance vs. hematocrit from assay chambers prepared with and without prothrombin reagents, derived from results of Example 1.

FIG. 12 illustrates the comparison of impedance vs. particle fraction from various sample dilutions of latex beads, derived from results of Example 2.

FIG. 13 illustrates the comparison of plots of log impedance vs. particle fraction from various sample dilutions of latex beads, derived from results of Example 2.

FIG. 14 illustrates the comparison of plots of impedance vs. particle fraction from various sample dilutions of Staphylococcus aureus cells coated with Protein A, derived from results of Example 3.

### DETAILED DESCRIPTION OF THE INVENTION

The present invention encompasses a sample analyzer system, device, test cards, methods and kits for performing multiple assays on fluid samples, including bodily fluids such as blood, plasma, serum, urine, cerebrospinal or cerebral spinal fluid, as well as non-bodily fluids, including water, liquid food/drink, commercial or medical products, or fluids derived from other sources. The analyzer system provides a device with disposable test cards for use at the point of care of a patient such as in a doctor's office, in the home, or elsewhere in the field. By providing an analyzer system kit with the device and disposable test cards with all of the needed reagents, the analyzer system can be reliably used outside of the laboratory environment, with little or no specialized training. Further, an analyzer system capable of performing multiple tests on small fluid samples streamlines the sample analysis process, reduces the cost and burden on medical or other personnel, and increases the convenience and compliance for the user, including those that require relatively frequent monitoring and/or analyses.

Accordingly, the analyzer system of the present invention provides methods and test cards configured for a variety of assays. For example, the analyzer system can concurrently determine the particle fraction and the change in viscosity of a sample such as coagulation rate and hematocrit of a whole blood sample using a single test card, and may be configured to compare the coagulation rate to positive control samples. The analyzer system of the present invention provides test cards, methods and a device for measuring prothrombin time (PT), activated partial prothrombin time (APTT), the activated clotting time (ACT), or the thrombin clotting time (TCT) of a sample. The analyzer system may also be used to detect the presence of an analyte in a fluid sample, such as blood, plasma, serum, urine, cerebrospinal or other bodily fluids, or non-bodily fluids. The analyzer system may also be used to determine the aggregation of particles in a fluid sample, including the aggregation of platelets, erythrocytes, or the aggregation of synthetic substrate, cellular, or microbial particles in immunological or analyte assays.

Electrical impedance, or simply impedance, is generally a measure of the opposition to a sinusoidal alternating electric current. Similarly, electrical resistance, or simply resistance, is typically a measure of the opposition to a DC electric current. While generally applied to a sinusoidally alternating current, impedance can be used for measuring the opposition to other alternating waveforms as well. Unlike electrical resistance, the impedance of an electric circuit can be a complex number, however, like resistance, the unit of impedance is typically the ohm. The impedance measurements of the present invention can be carried out at a various frequencies and voltages, and the resistance measurements can similarly be performed at various voltages.

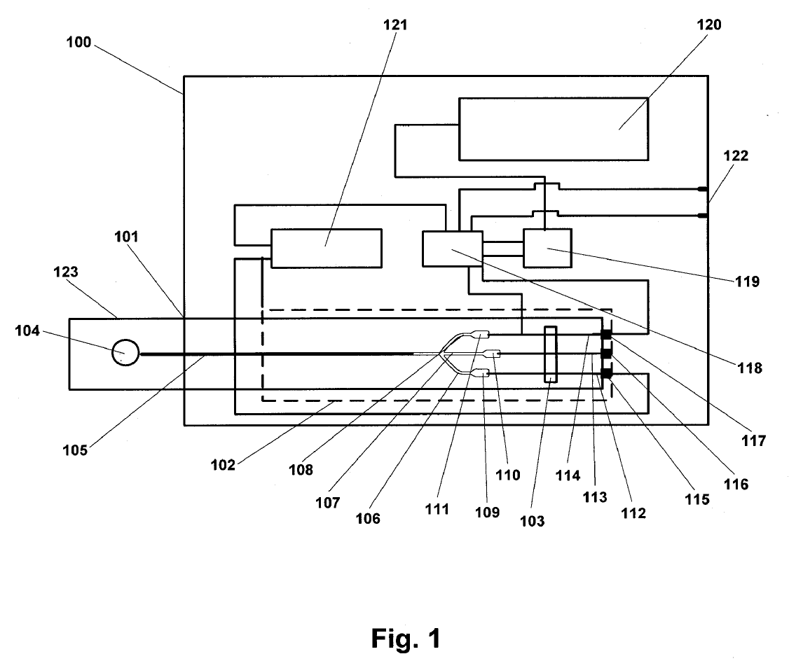

A device of the analyzer system is reusable and compact for hand-held operation or easy portability and is adapted to receive a disposal test card inserted therein. A representative example of the device of the present invention is illustrated in FIG. 1. The device includes a housing 100 having an inlet port 101 for receiving a disposable fluidic test card 123 for at least partial insertion therethrough. The test card 123 is preferably disposable after performing the desired assay. Test cards can include any assay strip, cartridge, adapted and configured to receive a fluid sample and support one or more reagents and electrodes as described herein, as well as any other device suitable to achieve that purpose available to a person of skill in the art. Additionally the test cards and/or the device itself may assume any convenient geometric shape as long as the electronics and chemistry described herein are cost effectively contained with acceptable performance.

In one embodiment, the device is configured such that all of the components are mounted in the interior space of the housing 100, including a power supply 121 to conduct the assay. Optionally, the device may provide a plug 122 for an AC adaptor. In this configuration, the test card 123 is adapted and configured to be inserted into the device and is positioned in thermal proximity to a heater 102 which is used to warm the sample on the test card to a target temperature. The target temperature can be pre-determined and can be above room temperature, i.e., greater than 35° C. Any conventional heater of the appropriate size and heating capacity for the anticipated sample size is suitable. Preferably, the heater is mounted between or below a substrate such as aluminum or other thermally conductive material for efficient and uniform transfer of heat to the test card. Preferably, the temperature is maintained at 37° C. so that the test results can readily compared to other standardized test results without interpolation.

A temperature sensor 103 is mounted in proximity to the detection or sampling area 109, 110, 111 where the sample is applied or transported to in order to detect the temperature of the system and provide ambient temperature information for calibration adjustment at temperature extremes. It is suitable to locate the temperature sensor 103 anywhere in or on the device. For example, the temperature sensor 103 may be located on the test card.

The power supply 121 has a lead from its negative pole connected to one side of an electrode pair 115, 116, and 117 and a lead from its positive pole for delivering AC or DC electric potential to the electrodes and detection of the impedance or resistance signal, which is relayed by electrical connection to an analog to digital converter 118 and display 120. A processor and memory component 119 is connected to the analog to digital converter 118 and the display 120. External ports 122 are connected to the analog to digital converter 118 for receiving assay calibration information, interfacing with a computer, or downloading test results. The distal end of the receiver electrodes 112, 113, and 114 that are in contact with corresponding contact pads 115, 116, and 117 of the measuring device which provide connection to the processor 119 and the power source 121. The connection between the electrodes 112, 113, and 114 and the contact pads 115, 116, and 117 is made when the test card 123 is inserted into the inlet port 101.

The processor 119 can be any common or custom integrated circuit with memory. The power supply 121 can be any convenient device including, but not limited to, a battery, AC adapter, or a solar cell. The display 120 preferably is a liquid crystal device LCD or any conventional, inexpensive display device. The number size in the display should be sufficiently large to allow most people to read the assay value, even if they have poor vision. The display height can be about 2.0 cm. The number of digits in the display can be anywhere from 1 to about 10 digits, however, a 3 to 5 digit display is usually sufficient. In addition to showing the assay result, the display may show messages relating to the assay result or processing or give error messages.

The converter 118 can include a multiplexer to integrate the signal from the electrodes and provide the digital signal to the processor 119. The processor can be used to count the time required for the integral to reach a fixed voltage comparator threshold. The time is proportional to the average signal over the sampling period.

The device can be of any convenient size with the optimal dimensions determined by several factors including convenience of use to the consumer. Preferably, the device has a volume range of about 5 cm³ to about 500 cm³.

As described above, the inserted test strip 123 is positioned in proximity of a heater 102. Thus, when in use, the sample is maintained at a target temperature. Alternatively, a heater can be included within the test card. The temperature of the sample is maintained constant by signaling from a temperature sensor 103 to the heater. The sensor is mounted in proximity to the detection area of the test strip, or it may be mounted anywhere on the device, and it provides temperature information for calibrating the device and ensuring that the assays are conducted at a predetermined constant temperature to eliminate interassay temperature variations.

When a fluid sample (e.g. blood sample) is applied to the sample well 104 of the test strip, it is transported through the microfluidic feeder channel 105 and capillary branch channels 106, 107 and 108 by capillary action, and is delivered to the reaction chambers 109, 110, and 111. The volume of blood that is applied to the test strip can be between 5 and 20 μL, or between 10 and 15 μL. Sample flow is stopped within each of the reaction chamber(s) by air vents 109, 110, and 111 that act as stop junctions.

Within the reaction chamber, the fluid sample reacts with the reagent(s) in the reaction chamber and bridges the electrodes, thereby changing the impedance or resistance between the electrodes and signaling the change to the measuring device, thereby initiating the assay. The electrodes are adapted to receive and pass a predetermined signal into the sample. The electrodes generate an electrical signal corresponding to the impedance or resistance of the sample. For example, the change in impedance or resistance is signaled through the distal end of the receiver electrodes 112, 113, and 114 that are in contact with corresponding contact pads 115, 116, and 117 of the measuring device to an analog to digital converter 118, which integrates the signal from the electrodes and conveys it to a processor and memory component 119, and a display 120. The device is driven by a source 121 that can be any power source such as a battery, AC adaptor, or a solar powered cell.

Typically, the processor 119 is adapted to have the capacity to either store a set of pre-programmed calibration information or have the capability to be programmed during device manufacturing. In the case of preprogrammed calibration, selection of appropriate information during manufacture is necessary and can be done by laser burning of a selection of circuit pathways or any convenient means. In the case of post-manufacture calibration, a method to load calibration data onto the chip is necessary, for example external ports 122. External calibration can be accomplished with external electrical contacts or may be done with a non-contact method using radio waves, magnetic fields, pulse light, laser or the like. The non-contact method of calibration may be more practical and efficient from a manufacturing viewpoint.

The processor 119 can also be configured to control the entire operation of the instrument including, but not limited to, turning the instrument on in response to insertion of a test card 123, providing electrical power or time signals; timing with an on-board clock, recording, and processing the instrument zero function; controlling any time delays or timed steps during reading; determining when the assay has stabilized; receiving and processing information from the temperature sensor; controlling application of electric potential to and receiving input from the test card; measuring the electrical properties of the sample and converting it to output, based on calibration information, which is relayed to the display unit and/or data port for output of the assay results, or optionally is stored in memory. The processor can further be adapted to determine if the assay reaction has occurred within the specified time, to a specified endpoint range or within a specified reaction rate range to control for inactive reagents. Any other electronic control checks can also be included. The processor 119 can be adapted to include codes that identify the assays of the test card. Additionally, the processor 119 can be adapted to contains a program which includes, but is not limited to, interpreting the current off the electrodes, relating the signal strength ratio to the reference strength, comparing the detected signal to stored calibration information, outputting assay results, identifying potential errors, and performing other quality control checks.

Examples of the information stored in the microprocessor 119 includes, but is not limited to, algorithms or calibration curves for the particle fraction, rate of coagulation, or analytes selected for analysis and other assay calibration information; reaction stabilization, endpoint, or rate information; and manufacturing lot information on each of the chemical reagents, detectors, LEDs, test cards, and other components used in the device.

With respect to calibration information, information would be stored such that a value of International Normalized Ratio (INR) may be given as a result of a coagulation test. PT test results can be converted to International Normalized Ratio (INR) values. The INR serves to eliminate interlaboratory differences in test results, which are caused by the use of thromboplastins with different sensitivities. Each thromboplastin is assigned an ISI based on comparison to an international reference thromboplastin from the World Health Organization (WHO). The INR is calculated by raising the prothrombin time ratio (PTR; the patient's prothrombin time divided by a reference normal prothrombin time) to the power of a coefficient known as the International Sensitivity Index (ISI). This coefficient relates the sensitivity for monitoring oral anticoagulation therapy of a given thromboplastin to the sensitivity of the WHO's reference preparation of thromboplastin, which is assigned an ISI of 1.0. Thromboplastins less sensitive than this international reference preparation of thromboplastin have proportionately higher ISI values. Thus Prothrombin Time is expressed as the INR ratio as:

INR=(PT ratio)^{ISI}, and PT ratio=Patient's PT/Mean Normal PT

The processor 119 can be adapted and configured to contain a program or analyzer adapted to, for example, interpret the current signals from the electrodes, relating the signal strength ratio to the reference strength, provide assay results, identify potential errors, and perform other quality control checks. Assay information can be relayed to the display 120 from the processor 119, which, in addition to showing the assay results, may display messages related to the processing functions, and messages relating to the assay results including error messages.

The particle fraction of a fluid sample may be determined by the methods of the present invention by measuring the impedance or resistance of the sample at one or more time points by the driver and receiver electrode pair of an assay chamber in comparison to a calibration standard curve. The aggregation of the particles of a fluid sample may be determined by the methods of the present invention by measuring the net difference in impedance or resistance of the sample in an assay chamber by measurements taken at the initiation and completion of aggregation promoted by assay reagents or by contact of the fluid sample in an assay chamber comprising a plurality of particles coated with reagents or antibodies. Alternatively, the aggregation of the particles of a fluid sample may be determined by the net difference in impedance or resistance of the sample, including the steps of taking a first measurement of impedance or resistance in an assay chamber without aggregation-promoting reagents and then comparison to one or more measurements taken in an assay chamber comprising reagents or a plurality of coated particles that initiate aggregation. The rate of coagulation or lysis of a sample may be measured by the methods of the present invention as a rate of change in impedance or resistance that is measured continuously by the driver and receiver electrode pair(s) of each reaction chamber, including the steps of: accelerating coagulation of the sample by chemically reacting the sample with at least one reagent to produce a rate of change in the impedance or resistance of the sample that can be detected and which correlates with a state of coagulation or lysis of the sample, measuring the impedance or resistance of the sample and generating a signal which correlates to a curve of the coagulation or lysis, and processing the signal into an output corresponding to the coagulation or lysis assay using assay calibration information.

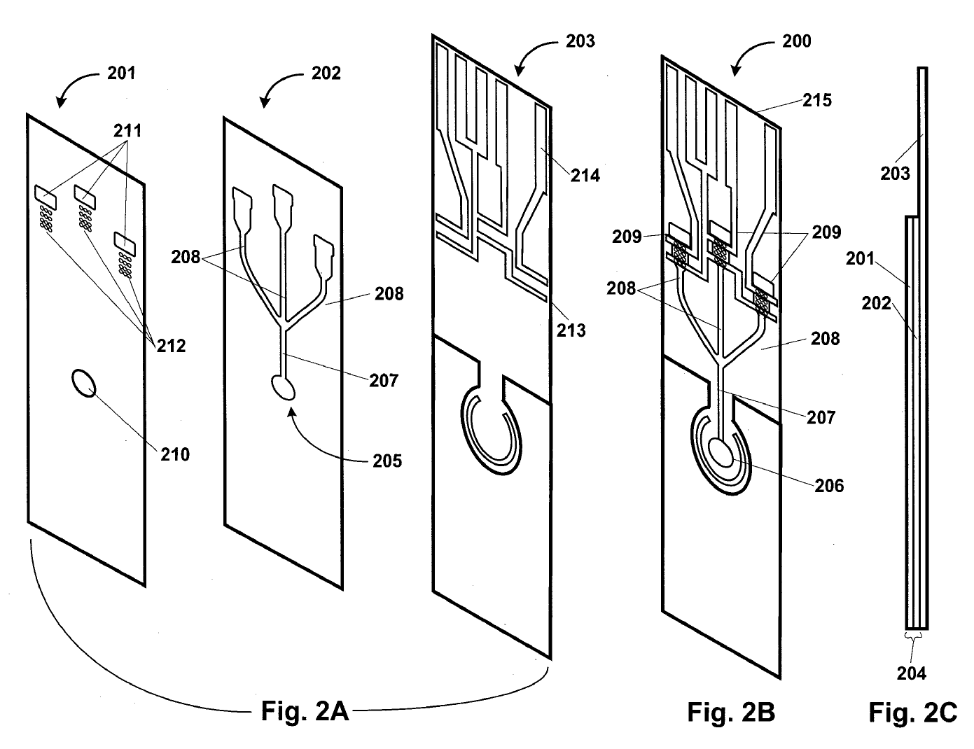

In one example, the fluidic test card of the present invention can be produced as a laminate having three layers 201, 202 and 203, as shown in the embodiment depicted in FIG. 2A, defining a surface for receiving the fluid sample and capillary channels for delivering the sample to assay chambers by capillary action. Layer 201 is the top layer; layer 202 is the middle layer, and layer 203 is the bottom electrode layer. The assembled strip 200 (FIG. 2B) is obtained by first assembling the top layer 201 and middle layer 202 to form a top-middle layer 204 (illustrated in FIG. 2C), and then combining the top-middle layer 204 with the electrode layer 203.

As illustrated in FIG. 2A, the fluidic path 205, which transports the sample from the sample well 206 through the microfluidic feeder 207 and branch channels 208 to the reaction chambers 209 of the assembled strip. The top layer 201 is perforated to provide a circular opening 210 that aligns with the portion of the microfludic path 205 that forms the sample well 206 in the assembled test strip 200. Additional cutouts of the top layer 201 can be made to provide the air vents 211, which act as stop junctions to halt the flow of fluid sample within the assay chamber during use of the strip. The top layer houses the reagents or compositions that enable the viscosity and/or analyte determination of a sample during use of the test strip. The reagents are deposited in the reagent areas 212 may be located below the cut of the air vent, and on the side of the top layer that is adjacent to the middle layer. The electrodes are formed on the electrode layer 203 by depositing a suitable inert conductor using a pump or any other means of deposition, such as vacuum or sputtering. Electrodes may also be produced by the printing of an ink using methods known in the art, such as bubble jet printing. Suitable conductor materials that can be used to form the electrodes include but are not limited to silver, carbon, gold or platinum. The electrodes have a proximal end 213 and a distal end 214, which respectively span the region of the electrode layer 203 that is complementary to that defining the reaction chamber 209 and the signaling edge 215 of the assembled test strip 200.

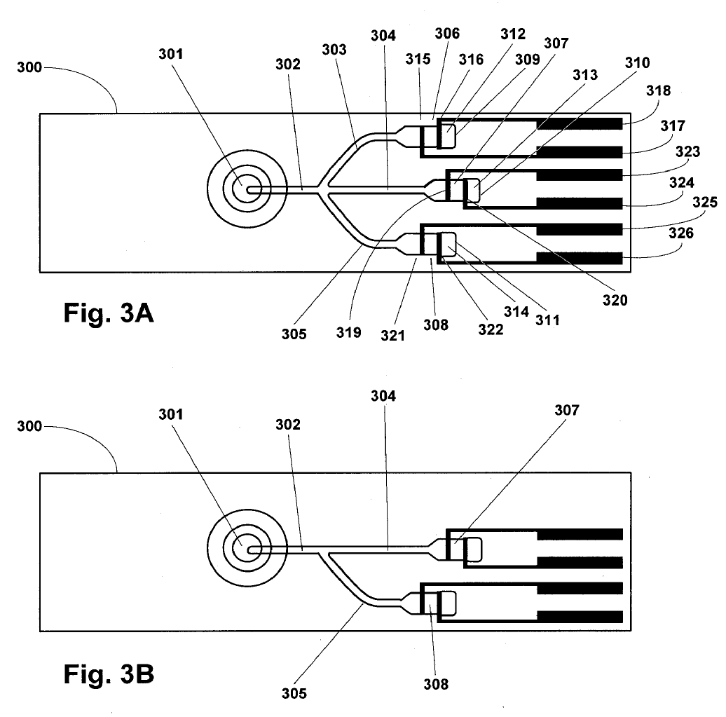

As shown in the embodiment of FIGS. 3A-B, the test card may be configured to have two or more assay chambers. Each assay chamber also contains a detector for detecting the impedance, resistance or change in impedance or resistance of the sample comprising a driver and a receiver electrode. The driver electrodes 317, 323 and 325 deliver an electric potential to the reaction chambers 306, 307, and 308, and the receiver electrodes 318, 324, and 326 deliver an electrical signal reflective of the electrical properties, i.e., impedance or resistance, of the sample present in the reaction chambers to a measuring device that translates the impedance signal into a digital output. In one embodiment, an AC electric potential is applied to the electrodes through the fluid sample and the measured impedance signal is impedance. The electric potential that is provided by the driver electrodes 317, 323 and 325 enters the reaction chambers via a proximal end 315, 319, and 321 of the driver electrodes. The proximal end 316, 320, 322 of the receiver electrodes 328 are positioned within the reaction chamber distal to the proximal ends of driver electrodes 315, 319 and 321. The assay chambers are typically configured to comprise an electroactive reagent to ensure electrical conductivity in the fluid sample. Electroactive species that can be used include but are not limited to ferricyanide, ferrocyanide, cadmium chloride, and methylviologen are most preferred electroactive species for use with the present invention.

Any of the test cards herein can be designed to be inserted into the measuring device of the invention such that the distal end of the electrode pair 112, 113, 114 on the test card 100 engages with and makes electrical contact with corresponding electrical contact points of the measuring device 115, 116, 117. Electrical parameters measured by the electrodes are then transmitted to the measuring device which is able to interpret the signal in order to give a result. The electrodes may be of any suitable shape or size and may be positioned within the reaction chamber(s) as pairs of driver 315 and receiver 316 electrodes, as shown in FIG. 3A.

The impedance or resistance signal that is detected and signaled by the receiver electrode(s) 318, 324 and 326 is indicative of the electrical properties of the sample in the reaction chamber(s) 306, 307 and 308. In some applications of the present invention, the electrical properties of the sample reflect a condition related to the particle fraction of the sample. In other applications, the electrical properties of the sample reflect the degree or ability of a sample to lyse or coagulate. In yet further applications, the electrical properties of the sample are used to detect the presence or absence of an analyte in the sample. In still further applications, the electrical properties of the sample are used to measure the aggregation of cells, particles, or substrates in the sample. Measurements of the impedance or resistance may be once, repeated, or can be taken continuously over a period of time to provide a measurement indicative of the condition that is being tested.

The test card 300 of the present invention can be designed to have various configurations. The test card comprises a sample well 301 that is open to the atmosphere. The sample well 301 is fluidly coupled to a microfluidic feeder channel 302 that is subdivided into two or more branch capillary channels. For example, in the test card configuration exemplified in FIG. 3A, feeder channel 302 is subdivided into three branch capillary channels 303, 304, and 305 that ends in different reaction chambers 306, 307, and 308.

The test card, or test strip, of the present invention provides that one or more assay chamber may be configured to contain assay reagents for the assay to be performed. Such assay reagents can be used to determine the condition of a sample. For example, in one configuration at least one of the assay chambers 307 of the test card is an assay reaction chamber that comprises reagents needed for the assay of a sample, one of the assay chambers 308 is a control reaction chamber that comprises reagents that induce a desired assay outcome of a sample to occur within a predetermined range, and one of the assay chambers 306 does not contain a reagent and is used optionally as a negative control or to measure the particle fraction by impedance only. It is understood that the assay and control reagents will vary depending on the type of test that is being performed. For example, control reagents may be included that induce the increased viscosity of a sample to occur within a predetermined range of time or promote the aggregation of a particle sample to within a predetermined percentage of the overall sample.

An advantage of the present invention is that test cards may be configured with different assay reagents provided or withheld within different assay reaction chambers to allow for the concurrent performance of two or more different assays using one test card. For example, in one test card configuration, hematocrit and coagulation rate test assays are each carried out simultaneously in a different reaction chamber of the test card. In a different configuration the hematocrit and coagulation rate assays are conducted in the same assay chamber while, optionally, positive control and delayed control coagulation assays are conducted in separate assay chambers. In a further configuration, the hematocrit assay may be conducted in one assay chamber, an analyte or immunologic assay in a second chamber, and a control assay in a third chamber. In other configurations, various permutations of the aforementioned assays may be incorporated in a particular test card.

The test card of the present invention may be configured to comprise one or more reagents that induce changes in viscosity in the fluid sample, such as coagulation or lysis. For example, different coagulation or lysis promoting reagents may be provided within different reaction chambers to allow for the simultaneous measurement of two or more different coagulation times, such as including reagents for PT and APPT assays. Typically, the reagent compositions for the specified test are applied to the test strip during the manufacturing process of the test strip and using various types of micro-dispensing techniques which include, but is not limited to, ink jet, striper and sprayer deposition methods, or dip coating, and air dried in situ during the manufacturing process.

In another example, the test card of the present invention comprises one or more assay reagents for detecting the presence or absence of an analyte, resulting in a change in impedance signal as a means of detecting the presence or absence of an analyte. In a further example, the assay chamber comprises particles coated with a chemical and/or immunologic agent to facilitate the aggregation of the particles and a resulting change in impedance signal as a means of detecting the presence or absence of an analyte, protein molecule, or a microbe. The use of such particles in an immunologic assay test card can applied to both an antigen and an antibody as targets for measurement. Such test cards would be employed in assays to determine, for example, the presence of specific circulating antibodies of interest, the detection of microbes, for ABO or other cell typing, detection and quantitation of analytes such as hormones, drugs, chemicals, proteins, blood factors, and the like. Examples of the assay or substrate particles of the present invention include, but are not limited to, latex beads, whole blood cells, erythrocytes, white blood cells, platelets, colloidal gold particles, magnetic particles, quantum dots, bacteria, viral particles, and liposomes. The analyte or immunologic reagent may further comprise a fluorescent tag that is activated or released as a result of the presence of an analyte, protein molecule, or a microbe.

In another configuration, at least one of the assay chambers of the test card does not comprise assay reagents and is used for determination of the particle fraction of the sample, pre-existing aggregation of particles within a fluid sample, or, optionally, as a control assay for comparison to an assay reaction chamber of the test card comprising reagents.

Typically, the reagent compositions for the specified test are applied to the test strip during the manufacturing process of the test strip and using various types of micro-dispensing techniques which include, but is not limited to, ink jet, striper and sprayer deposition methods, or dip coating, and air dried in situ during the manufacturing process.

The physical condition of a fluid sample may be measured based on its electrical conductivity. The analytical system of the present invention measures impedance of an introduced fluid sample at one or more time points in order to detect the physical condition of the fluid sample. Preferably, the device of the present invention measures impedance by applying AC potential to a fluid sample introduced into a disposable test card and detecting the resulting impedance signal from one or more assay chambers at one or multiple time points or continuously for a predetermined period of time. By such methods, the device of the present can process and interpret the impedance signal, resulting in a signal indicative of the change and an output of assay results for a number of pre-configured assays. For example, the relationship between the changing impedance of a coagulating or clot retracting blood sample has been described in Ur, A., “Analysis and Interpretation of the Impedance Blood Coagulation Curve,” American Journal of Clinical Pathology 67:470-476, 1977, but is dependant on a number of variables, such as chamber volume, sedimentation of blood during the assay, and presence of presence of anticoagulants.

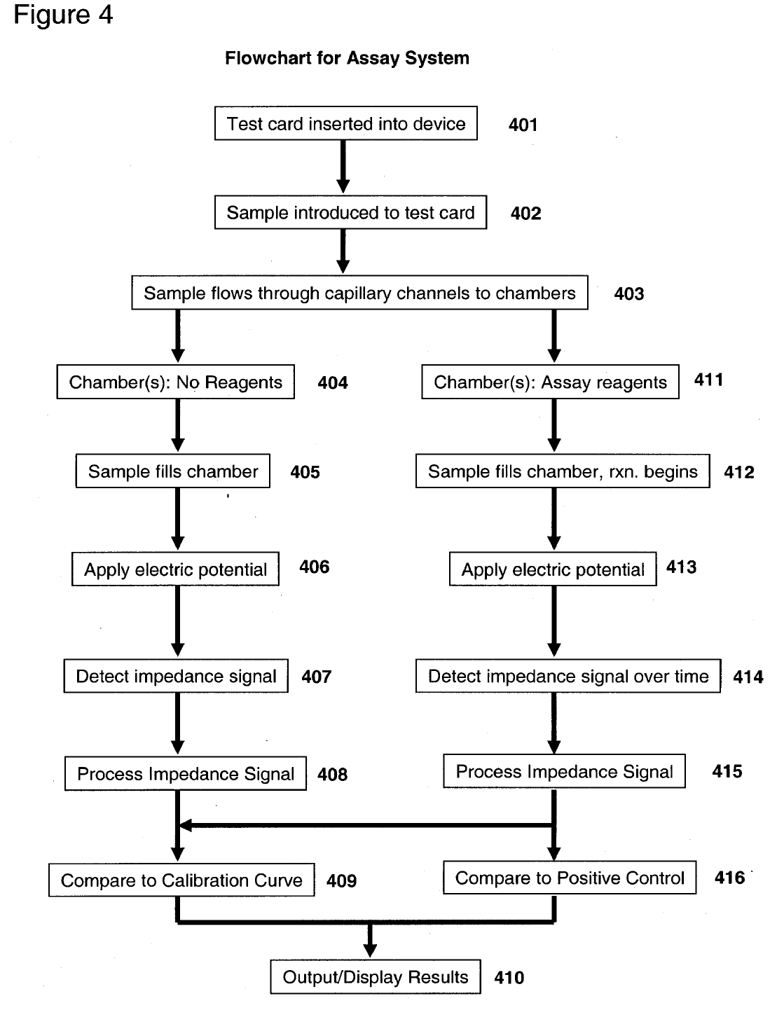

Use of impedance or resistance for assay determinations by the methods of the present invention can be understood with reference to an illustration of exemplary steps shown in FIG. 4. After insertion of a test card into the measuring device 401, a fluid sample is introduced into the test card 402 where the fluid sample flows to one or more assay chambers that, depending on the configuration of the test card, contain no assay reagents 404 or one or more assay reagents 411. A chamber configured with no assay reagents can be used, for example, for particle fraction determination, e.g., hematocrit. In such an assay, the fluid sample flows by capillary action and fills the sample chamber 405. The bridging of the electrodes by the fluid sample allows the device to apply an electric potential 406 to the electrodes and the impedance or resistance signal is detected 407 and sent to the measuring device where it is processed 408 and compared to a stored reference calibration curve 409. The results of the assay, plus any error messages; e.g., an out of control assay, would be output to the display 410.

In an alternative configuration, an assay is performed by comparing the impedance or resistance of a portion of a fluid sample that is reacted with chemical or immunological reagents to the impedance or resistance of a portion that is not reacted; e.g., platelet aggregation assay. In such a representative assay, one portion of the fluid sample containing platelets would flow to an assay chamber with no reagents and electrical properties would be measured by the steps outlined above 401-407. A second portion of the fluid sample would flow to a chamber and mix with assay reagents 411 that, in the representative example, would promote the aggregation of platelets 412. At one or more predetermined time points, the device would apply an electric potential to electrodes of the reagent-containing chamber 413 and the impedance or resistance signal would be detected 414 and be processed 415. The processor would then compare the impedance signal to that of the sample chamber not containing reagents 408, and the net impedance signal would be compared to a reference calibration curve 409, with the results, plus any error messages; e.g., an out of control assay, output to the display 410.

In yet another configuration, the assay is performed by comparing the impedance of a portion of a fluid sample that is reacted with chemical or immunological reagents to the impedance of a portion that is reacted with chemical or immunological reagents that include a positive control; e.g., a prothrombin time. In such an example, one portion of the fluid sample would flow to a chamber and mix with assay reagents 411 that, in the representative example, would promote the coagulation of the sample 412 while another portion would flow to a chamber that includes chemical reagents to promote the coagulation of the sample as a positive control. At multiple time points or continuously, the device would apply an electric potential to electrodes of the reagent-containing chambers 413 and the impedance or resistance signals would be detected 414 and be processed 415 to: (1) determine the endpoint of the prothrombin time; and (2) compare the assay prothrombin time to that of the positive control 416 to determine if the assay is within control parameters. The results of the assay, plus any error messages; e.g., an out of control assay, would be output to the display 410.

Use of the test cards with the device of the invention can be understood with reference to an illustration of the elements of the measurement device shown in FIG. 1.

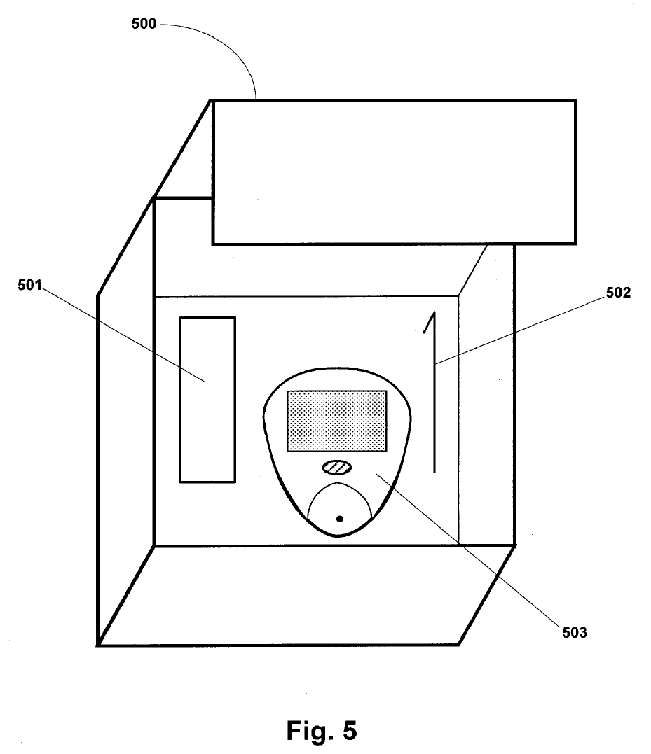

Also included within the scope of the present invention are kits providing the device and one or more of the test cards of the invention. For example, as shown in FIG. 5, in such a kit 500 one configuration can comprise the measuring device of the invention 503 and one or more test cards 501 with assay chambers configured to perform hematocrit and one or more coagulation assays such as prothrombin time or activated partial prothrombin time. In an alternative configuration, the kit can comprise the measuring device and one or more test cards with assay chambers configured to perform analyte or immunologic assays for detection of therapeutic drugs, measure or detect antigens, specific antibodies, or other blood components. The kits of the present invention will contain instructions 502 for using the device and test cards for the methods of the analyzer system; such instructions can be in the form of printed, electronic, visual, and or audio instructions.

Having generally described the present invention, a further understanding can be obtained by reference to the following specific examples, which are provided herein for purposes of illustration only and are not intended to be limiting of the present invention.

### EXAMPLES

### Example 1

### Determination of Hemotocrit by Measurement of Impedance

The analyzer system, device and methods of the present invention were utilized to demonstrate the determination of the hematocrit of a blood sample over a range of hematocrit percentage values.

Blood samples were obtained by venipuncture and were collected in tubes containing 3.2% citrate (Becton Dickinson Vacutainer). The baseline hematocrit of each sample was determined by measurement of the hemoglobin with a HemoCue Homoglobin Photometer (HemoCue AB, Angelholm, Sweden) following the procedures provided by the manufacturer, with the hematocrit value derived mathematically by multiplying the hemoglobin concentration (g/dL) by three. Samples with the derived hemocrit value were then used to prepare test samples of varying hematocrit value by centrifuging the samples and removing a defined volume of plasma (thereby increasing the hematocrit upon remixing) or by dilution of a sample of known hematocrit by autologous plasma. The samples thusly prepared were reassayed for hematocrit by measurment of the hemoglobin content of the prepared materials as described above. Thus, samples with a range of hematocrit values were created for assay by the device of the invention.

The prepared samples were measured in test cards designed for use in a HemoSense INRatio Monitor (Hemosense, Inc., San Jose, Calif.). In some cases the test cards were used in the device without incorporation of coagulation reagents. In such cases, only the hematocrit-dependant changes in impedance were measured. In other cases the test cards contained reagents to promote coagulation for prothrombin and INR assays of whole blood. In the latter case, a 2.5 μL aliquot of 350 mM Ca⁺² was added to the sample to overcome the effects of the citrate anticoagulant.

For each assay, a sample of approximately 15 μL was applied to the sample well position of a test card inserted into the HemoSense INRatio Monitor device and was taken by capillary action into the assay chambers that have a volume of approximately 1 μL. The impedance values were then measured by the device.

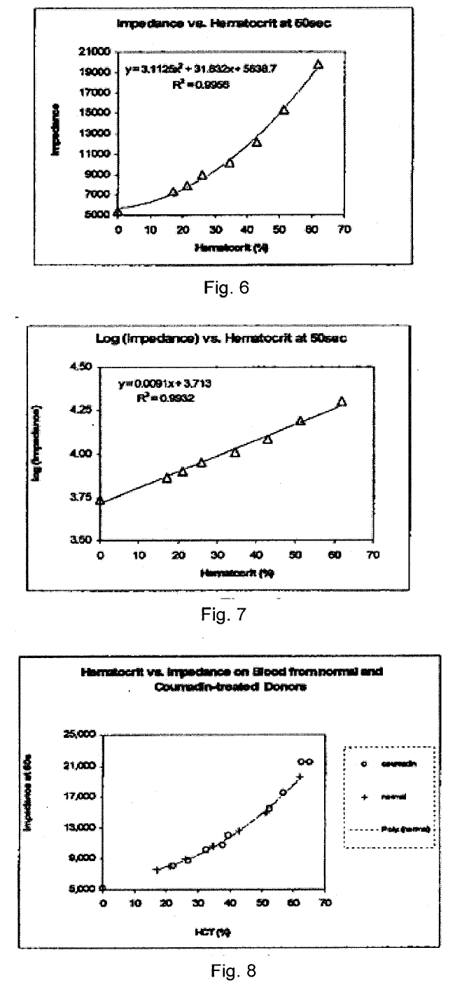

In FIG. 6, the impedance value of the samples measured at 50 seconds was plotted vs. the known hematocrit values. The response can readily fit into a second order polynomial equation, with an R² value of 0.9956. Impedance values have been measured at 6, 20, and 50 seconds after introduction of sample, and while the impedance values drop slightly, the correlation to hematocrit remains. When the assay was performed using a test card with prothrombin reagents and the results were plotted as the logarithm of the impedance vs. hematocrit (FIG. 7), the response is linear, with an R² value of 0.9932, indicating an excellent correlation between the two parameters under these conditions.

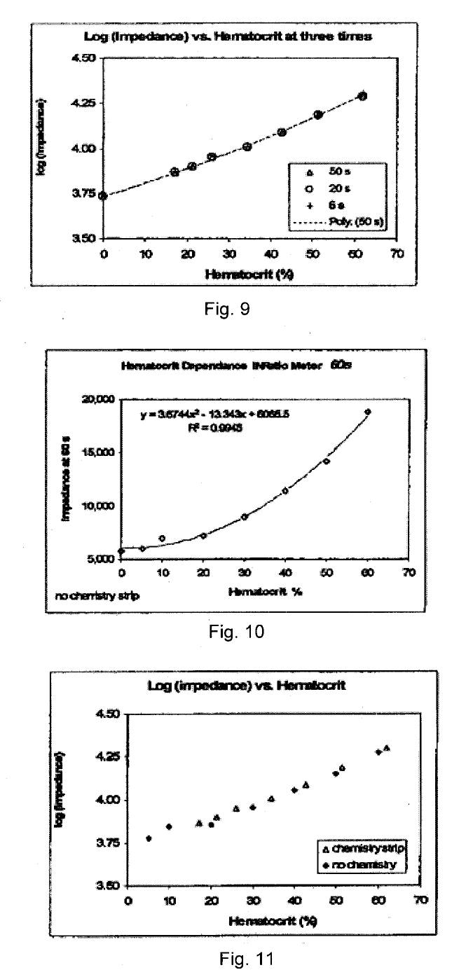

Assays were performed to assess the effect of anticoagulant therapy in patients on the performance of the hematocrit assay. Blood was obtained from a subject on Coumadin. Prothrombin time assays confirmed that the rate of coagulation was approximately twice that of an untreated control subject (data not shown). Samples from the Coumadin-treated and untreated subject were prepared over a range of known hematocrit values as described above and used in the assay system. The results from samples measured in the middle test card cell, shown in FIG. 8, demonstrate that treatment with the anticoagulant does not affect the performance of the hematocrit assay, in comparison to untreated subject sample. Results were similar for samples in the other two test card chambers. Similarly, the results for the Coumadin-treated subject sample were similar when measured over three intervals and performed in assay chambers containing reagents that promote coagulation (FIG. 9). Similarly, FIG. 10 shows results from the same samples tested in chambers without coagulation, with a R² value of 0.9945. In FIG. 11, the plots compare log impedance vs. hematocrit in assays performed in assay chambers containing coagulation reagents and chambers with no reagents, and the response profiles are similar.

Overall, the results demonstrate that the hematocrit of a blood sample can be determined using impedance according to the methods of the invention, and that the assay can be performed in assay chambers containing coagulation promoting reagents without affecting the performance of the assay.

### Example 2

### Determining Particle Fraction of Fluids Containing Synthetic Particles

Using the general methods and approaches described herein, the analyzer system, device and methods of the present invention were utilized to demonstrate the determination of the particle fraction of a fluid samples over a range of particle fraction percentage values.

Latex particles of approximately 0.9 μM diameter with a carboxylate-modified surface (Sigma Chemical Co., St. Louis, Mo.) were used for all assays. The particles were washed in 0.075M NaCl solution and centrifuged, and then suspended in an equal volume of the NaCl solution to create a 50% particle suspension. The stock suspension was then diluted using the NaCl solution to create a range of particle suspensions for assay. Aliquots of 10 μL of the suspensions were then introduced into a test card without coagulation reagents and the impedance signal determined at a 20 second interval after introduction to the test card.

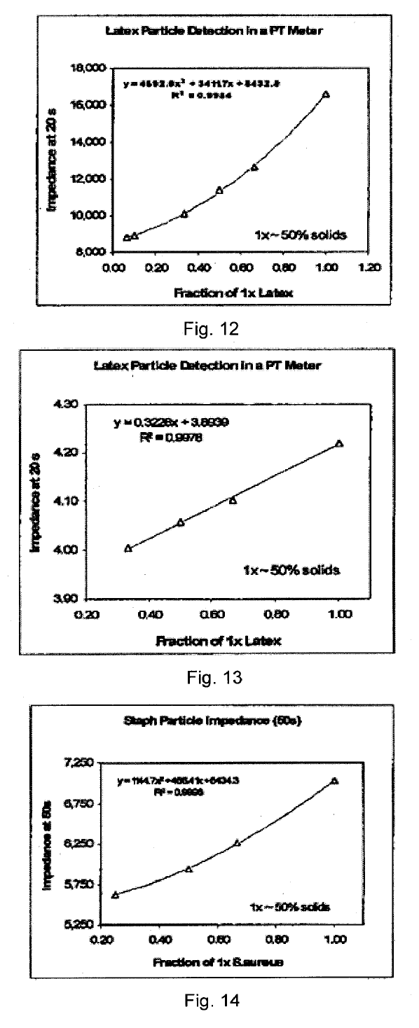

The assay results from one experiment were plotted in FIG. 12 as the impedance vs. the relative dilution of the stock suspension of particles. The plot shows a linear response with an R² value of 0.9978, indicating a high degree of correlation between impedance and particle fraction. In another experiment, the assay results were plotted as the log impedance vs. the relative dilution of the stock suspension of particles, and, as shown in FIG. 13, had an R² value of 0.9978; again showing excellent correlation.

### Example 3

### Determining Particle Fraction of Fluid Containing Microbial Particles

Using the general methods and approaches described herein, the analyzer system, device and methods of the present invention were utilized to demonstrate the determination of the particle fraction of a fluid samples over a range of particle fraction percentage values.

A non-viable preparation of Staphilococcus aureus particles linked to protein A, of approximately 0.8 μM diameter (Sigma Chemical Co., St. Louis, Mo.) were used for all assays. The particles were washed in 0.075M NaCl solution and centrifuged, and then suspended in an equal volume of the NaCl solution to create a 50% particle suspension. The stock suspension was then diluted using the NaCl solution to create a range of particle suspensions for assay. Aliquots of 10 μL of the suspensions were then introduced into a test card without coagulation reagents and the impedance signal determined at a 50 second interval after introduction to the test card.

Impedance was plotted vs. the fraction of the stock preparation, as shown in FIG. 14. The data show a curvilinear response with an R² value of 0.9996. Accordingly, the data demonstrate that there is a high degree of correlation between impedance and particle fraction.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for determining a particle fraction of a fluid sample comprising the step of: measuring impedance or resistance of said fluid sample, wherein said fluid sample volume is up to 20 μL.

2. The method of claim 1, further comprising the step of applying an AC or DC electric potential to said fluid sample.

3. The method of claim 1, wherein said particle fraction is hematocrit.

4. The method of claim 1, wherein said measuring step comprising the step of correlating said impedance measurement to a calibration standard curve to produce an impedance correlation.

5. The method of claim 4, wherein said impedance correlation is further processed into an output result corresponding to the particle fraction of said fluid sample.

6. The method of claim 1, wherein said fluid sample comprises a body fluid.

7. The method of claim 6, wherein said body fluid comprises blood.

8. The method of claim 1, wherein said fluid sample is of a non-bodily fluid.

9. The method of claim 1, wherein said fluid sample comprises water.

10. A method for determining particle fraction of a fluid sample comprising the step of: applying said fluid sample to a chamber having volume up to 2 μL; and measuring an impedance or resistance of said fluid sample in said chamber.

11. The method of claim 10, further comprising the step of applying an AC or DC electric potential to said fluid sample.

12. The method of claim 10, wherein said particle fraction is hematocrit.

13. The method of claim 10, wherein said measuring step further comprises correlating said impedance or resistance measurement to a calibration standard curve to produce an impedance or resistance correlation.

14. The method of claim 13, further comprising processing said impedance or resistance correlation into an output result corresponding to the particle fraction of said fluid sample.

15. The method of claim 10, wherein said fluid sample comprises a body fluid.

16. The method of claim 15, wherein said body fluid comprises blood.

17. The method of claim 10, wherein said fluid sample is a non-bodily fluid.

18. The method of claim 10, wherein said fluid sample is water.

19. A method for determining particle fraction of a fluid sample comprising the step of measuring electrical properties of said fluid sample, wherein said measuring step is used to determine said particle fraction, and wherein said fluid sample volume is up to 2 μL.

20. The method of claim 19, further comprising applying AC electric potential to said fluid sample.

21. The method of claim 19, further comprising applying DC electric potential to said fluid sample.

22. The method of claim 19, wherein said particle fraction is hematocrit.

23. The method of claim 19, wherein said measuring step further comprises:
(a) measuring impedance at one or more time points of said fluid sample and correlating said impedance to a particle fraction calibration standard curve to produce a first impedance correlation;
(b) measuring rate of change of impedance over time of said fluid sample and correlating said impedance to a coagulation calibration standard curve to produce a second impedance correlation; and
(c) processing said first and second impedance correlations into output results corresponding to the particle fraction and viscosity of said fluid sample.

24. The method of claim 19, wherein said fluid sample comprises a body fluid.

25. The method of claim 24, wherein said body fluid comprises blood.

26. The method of claim 19, wherein said chamber comprises one or more reagents for accelerating the coagulation of a fluid sample upon contact with said fluid sample.

27. The method of claim 26, wherein said reagents comprise reagents for prothrombin time, activated clotting time, activated partial prothrombin time, or thrombin clotting time.

28. A method of determining particle fraction and viscosity of a fluid sample comprising the steps of:
a) applying said fluid sample to a first chamber having volume up to 2 μL and a second chamber having volume up to 2 μL wherein the first chamber further comprises one or more reagents for accelerating a coagulation of a fluid sample upon contact with said fluid sample;
b) measuring impedance of said fluid sample at one or more time points in the second chamber and correlating the impedance to a particle fraction calibration standard curve to produce a first impedance correlation;
c) measuring a rate of change of measured impedance over time of said fluid sample in said first chamber and correlating said impedance to a rate of coagulation calibration standard curve to produce a second impedance correlation; and
d) processing said first and second impedance correlations into output results corresponding to a particle fraction and viscosity of said fluid sample.

29. The method of claim 28, further comprising applying AC electric potential to said fluid sample.

30. The method of claim 28, wherein said particle fraction is hematocrit.

31. The method of claim 28, wherein said fluid sample comprises a body fluid.

32. The method of claim 31, wherein said body fluid comprises blood.

33. A method for determining the amount or presence of analyte in a fluid sample comprising measuring change of impedance or resistance of said fluid sample in a chamber wherein chamber comprises a binding moiety that selectively binds to said analyte and wherein volume of said chamber is up to 2 μL.

34. The method of claim 33, wherein said determining step comprises
a) applying AC or DC electric potential to said chamber;
b) measuring impedance or resistance at two or more intervals;
c) correlating said change of impedance or resistance to an analyte calibration standard curve; and
d) processing said impedance correlations into output results corresponding to the amount or presence of said analyte of said fluid sample.

35. The method of claim 33, wherein said analyte is mammalian cells, microbial cells, drugs, chemical compounds, hormones, proteins, and immunoglobulins.

36. The method of claim 35, wherein said binding moiety is immunoglobulins, monoclonal antibodies, avidin, compromised avidin, streptavidin, lectins, protein A, haptens, biotin, iminobiotin, or sugars.

37. The method of claim 35, wherein said binding moiety is coupled to a substrate.

38. The method of claim 37, wherein said substrate is latex beads, whole blood cells, erythrocytes, white blood cells, platelets, colloidal gold particles, magnetic particles, quantum dots, bacteria, viral particles, or liposomes.

39. A method for determining the amount or presence of analyte in a fluid sample, wherein said determining step comprises:
a) applying said fluid sample to two chambers, said chambers having volume up to 2 μL, wherein a first chamber comprises a binding moiety for agglutinating an analyte in a fluid sample upon contact with said fluid sample and a second chamber does not comprise said binding moiety;
b) applying AC or DC electric potential to said first and second chambers;
c) measuring the difference in impedance or resistance of said fluid sample in said first and second chambers at one or more intervals;
d) correlating said difference in impedance or resistance to an analyte calibration standard curve to produce an impedance correlation; and
e) processing said impedance or resistance correlation into output results corresponding to the amount or presence of said analyte of said fluid sample.

40. The method of claim 39, wherein said analyte is mammalian cells, microbial cells, drugs, chemical compounds, hormones, proteins, and immunoglobulins.

41. The method of claim 39, wherein said binding moiety is immunoglobulins, monoclonal antibodies, avidin, compromised avidin, streptavidin, lectins, protein A, haptens, biotin, iminobiotin, or sugars.

42. The method of claim 39, wherein said binding moiety is coupled to a substrate.

43. The method of claim 42, wherein said substrate is latex beads, whole blood cells, erythrocytes, white blood cells, platelets, colloidal gold particles, magnetic particles, quantum dots, bacteria, viral particles, or liposomes.

44. A device for determining particle fraction, amount or presence of an analyte, and viscosity of a fluid sample comprising;
a) a detection unit adapted and configured to apply AC electric potential to said fluid sample and measure one or more impedance signals of said fluid sample;
b) a processor unit electrically connected to said detection unit adapted and configured to receive and convert said impedance signals into output results corresponding to said particle fraction, amount or presence of said analyte, or viscosity of said fluid sample.

45. The device of claim 44, wherein said processor unit separately determines said particle fraction, amount or presence of said analyte, or viscosity of said fluid sample from said impedance signals using assay calibration standard curve information stored in the processor.

46. The device of claim 44, wherein said particle fraction is hematocrit.

47. The device of claim 44, wherein said particle fraction is aggregated particles.

48. The device of claim 44, wherein said analyte is mammalian cells, microbial cells, drugs, chemical compounds, hormones, proteins, and immunoglobulins.

49. The device of claim 48, wherein said viscosity is rate of coagulation.

50. A test card for determining a condition of a fluid sample comprising: an inlet port for receiving a fluid sample of less than 20 μL fluidly connected to two or more capillary channels wherein each capillary channel terminates in a fixed volume chamber having a volume up to 2 μL.

51. The test card of claim 50, wherein each of said chambers further comprises two electrodes for measurement of impedance in said fluid sample.

52. The test card of claim 50, wherein one or more said chambers further comprises one or more reagents for accelerating the coagulation of said fluid sample upon contact with said fluid sample.

53. The test card of claim 50, wherein one or more said chambers further comprises one or more binding moieties that selectively binds to an analyte.

54. The test card of claim 53, wherein said binding moiety is immunoglobulins, monoclonal antibodies, avidin, compromised avidin, streptavidin, lectins, protein A, haptens, biotin, iminobiotin, or sugars.

55. The test card of claim 54, wherein said binding moiety is coupled to a substrate.

56. The test card of claim 55, wherein said substrate is latex beads, whole blood cells, erythrocytes, white blood cells, platelets, colloidal gold particles, magnetic particles, quantum dots, bacteria, viral particles, or liposomes.
